SEQUENCE LISTING (47 sequences)

SEQ ID NO: 1 - DNA, 18 bp, Artificial Sequence
  Oligonucleotide
cactaccctctcccatac

SEQ ID NO: 2 - DNA, 18 bp, Artificial Sequence
  Oligonucleotide
cactaccctctcctatac

SEQ ID NO: 3 - DNA, 19 bp, Artificial Sequence
  Oligonucleotide
ccaccaccctctcccatac

SEQ ID NO: 4 - DNA, 19 bp, Artificial Sequence
  Oligonucleotide
ccacttccctctcccatac

SEQ ID NO: 5 - DNA, 19 bp, Artificial Sequence
  Oligonucleotide
ccactaccctctcccgtac

SEQ ID NO: 6 - DNA, 19 bp, Artificial Sequence
  Oligonucleotide
ccactaccctctaccatac

SEQ ID NO: 7 - DNA, 20 bp, Artificial Sequence
  Oligonucleotide
tatctgaccgtcccaggtta

SEQ ID NO: 8 - DNA, 18 bp, Artificial Sequence
  Oligonucleotide
ccctctgatgggtaggtt

SEQ ID NO: 9 - DNA, 18 bp, Artificial Sequence
  Oligonucleotide
ccctctgatgggcaggtt

SEQ ID NO: 10 - DNA, 20 bp, Artificial Sequence
  Oligonucleotide
taggtgttgttagcatttcg

SEQ ID NO: 11 - DNA, 18 bp, Artificial Sequence
  Oligonucleotide
cactcctctttttccggt

SEQ ID NO: 12 - DNA, 19 bp, Artificial Sequence
  Oligonucleotide
ccacttctctttttccggt

SEQ ID NO: 13 - DNA, 19 bp, Artificial Sequence
  Oligonucleotide
ccactcttctttttccggt

SEQ ID NO: 14 - DNA, 19 bp, Artificial Sequence
  Oligonucleotide
ccactcttcttttcccggt

SEQ ID NO: 15 - DNA, 20 bp, Artificial Sequence
  Oligonucleotide
cacacaatcgtaacatccta

SEQ ID NO: 16 - DNA, 19 bp, Artificial Sequence
  Oligonucleotide
agggatgaactttccactc

SEQ ID NO: 17 - DNA, 18 bp, Artificial Sequence
  Oligonucleotide
ccactcattttcttccgg

SEQ ID NO: 18 - DNA, 18 bp, Artificial Sequence
  Oligonucleotide
cccccgcttgagggcagg

SEQ ID NO: 19 - DNA, 18 bp, Artificial Sequence
  Oligonucleotide
cctcttttcccggtggag

SEQ ID NO: 20 - DNA, 19 bp, Artificial Sequence
  Oligonucleotide
cctctttttccggtggagc

SEQ ID NO: 21 - DNA, 19 bp, Artificial Sequence
  Oligonucleotide
cactcctcttttccaatga

SEQ ID NO: 22 - DNA, 18 bp, Artificial Sequence
  Oligonucleotide
cactcctcttacttggtg

SEQ ID NO: 23 - DNA, 20 bp, Artificial Sequence
  Oligonucleotide
taggtgccagtcaaattttg

SEQ ID NO: 24 - DNA, 18 bp, Artificial Sequence
  Oligonucleotide
ccccttctgatgggcagg

SEQ ID NO: 25 - DNA, 18 bp, Artificial Sequence
  Oligonucleotide
ccccctctgatgggcagg

SEQ ID NO: 26 - DNA, 18 bp, Artificial Sequence
  Oligonucleotide
cgacttcgcaactcgttg

SEQ ID NO: 27 - DNA, 18 bp, Artificial Sequence
  Oligonucleotide
cgacttcgcgactcgttg

SEQ ID NO: 28 - DNA, 18 bp, Artificial Sequence
  Oligonucleotide
cgagttcgcaactcgttg

SEQ ID NO: 29 - DNA, 16 bp, Artificial Sequence
  Oligonucleotide
gacccccttgccgaaa

SEQ ID NO: 30 - DNA, 18 bp, Artificial Sequence
  Oligonucleotide
atgaccccctagccgaaa

SEQ ID NO: 31 - DNA, 20 bp, Artificial Sequence
  Oligonucleotide
ggcacaacctccaagtcgac

SEQ ID NO: 32 - DNA, 20 bp, Artificial Sequence
  Oligonucleotide
ggacaaccagcctacatgct

SEQ ID NO: 33 - DNA, 18 bp, Artificial Sequence
  Oligonucleotide
acaagactccagcctgcc

SEQ ID NO: 34 - DNA, 22 bp, Artificial Sequence
  Oligonucleotide
caggcggtctatttaacgcgtt

SEQ ID NO: 35 - DNA, 20 bp, Artificial Sequence
  Oligonucleotide
ggcacaacctccaaatcgac

SEQ ID NO: 36 - DNA, 20 bp, Artificial Sequence
  Oligonucleotide
ggccacaacctccaagtaga

SEQ ID NO: 37 - DNA, 18 bp, Artificial Sequence
  Oligonucleotide
accacactccagcctgcc

SEQ ID NO: 38 - DNA, 18 bp, Artificial Sequence
  Oligonucleotide
acaagactctagcctgcc

SEQ ID NO: 39 - DNA, 20 bp, Artificial Sequence
  Oligonucleotide
ggcggtcgatttaacgcgtt

SEQ ID NO: 40 - DNA, 20 bp, Artificial Sequence
  Oligonucleotide
ggcggtctacttaacgcgtt

SEQ ID NO: 41 - DNA, 20 bp, Artificial Sequence
  Oligonucleotide
ggcggtctatttaatgcgtt

SEQ ID NO: 42 - DNA, 21 bp, Artificial Sequence
  Oligonucleotide
agctccggaagccactcctca

SEQ ID NO: 43 - DNA, 18 bp, Artificial Sequence
  Oligonucleotide
ggaacaacctccaagtcg

SEQ ID NO: 44 - DNA, 18 bp, Artificial Sequence
  Oligonucleotide
gccacaacctccaagtag

SEQ ID NO: 45 - DNA, 18 bp, Artificial Sequence
  Oligonucleotide
atggccccctagccgaaa

SEQ ID NO: 46 - DNA, 19 bp, Artificial Sequence
  Oligonucleotide
gatgaccccctagccgaaa

SEQ ID NO: 47 - DNA, 20 bp, Artificial Sequence
  Oligonucleotide
aaccttgcggccgtactccc

(19) United States
(12) Patent Application Publication
(10) Pub. No.: US 2005/0064444 A1
(43) Pub. Date: Mar. 24, 2005

(54) Method for specific fast detection of relevant bacteria in drinking water

(76) Inventors: Claudia Beimfohr, Munchen (DE); Jiri Snaidr, Grossinzemoos (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/742,649

(22) Filed: Dec. 18, 2003

Related U.S. Application Data
(63) Continuation of application No. PCT/EP02/06809, filed on Jun. 19, 2002.

(30) Foreign Application Priority Data
Jun. 19, 2001 (DE) 10129 411.5
Dec. 11, 2001 (DE) 10160 666.4

(51) Int. Cl. C12Q 1/68; C07H 21/04

(52) U.S. Cl. 435/6; 536/23.7

(57) ABSTRACT

The invention relates to a method for detecting bacteria in drinking water and surface water, especially a method for simultaneous specific detection of bacteria from the Legionella species and the Legionella pneumophila species by in situ hybridization. The invention also relates to a method for specific detection of faecal streptococci by in situ-hybridization and a method for simultaneous specific detection of coliform bacteria and bacteria of the Escherichia coli species, in addition to corresponding oligonucleotide probes and kits enabling said inventive method to be carried out.

### CROSS REFERENCE TO RELATED APPLICATION

The present application is a continuation of PCT application Serial No. PCT/EP02/06809, filed Jun. 19, 2002, entitled “METHOD FOR SPECIFIC FAST DETECTION OF RELEVANT BACTERIA IN DRINKING WATER,” the disclosure of which is incorporated herein by reference in its entirety; which claims priority from German Patent Application Serial Nos. 101 29 411.5, filed Jun. 19, 2001 and 101 60 666.4, filed on Dec. 11, 2001, the disclosure of each of which is incorporated herein by reference in its entirety.

### BACKGROUND OF THE INVENTION

1. Field of the Invention

The invention relates to a method for detecting bacteria in drinking water and surface water, particularly a method for simultaneous specific detection of bacteria from the genus Legionella and the species Legionella pneumophila by in situ hybridization as well as a method for specific detection of faecal streptococci by in situ hybridization as well as a method for simultaneous specific detection of coliform bacteria and bacteria of the species Escherichia coli as well as corresponding oligonucleotide probes and kits enabling the said inventive methods to be carried out.

2. Description of the Related Art

Legionella are Gram-negative, non-sporogenous rod-like bacteria with a length of 0.5-20 μm and a diameter of 0.3-0.9 μm. They are motile because of their polar flagellation with one to three flagella. Legionella are ubiquitous inhabitants of wet soil as well as all non-marine aquatic habitats. Ideal conditions for their propagation are temperatures between 25° C. and 55° C. Consequently, they can also be found in habitats created by humans, such as for instance warm and cold water installations, cooling towers of air conditioning systems and water humidifiers. As intracellular parasites of amoebae and ciliates, they can also survive unfavorable living conditions, such as for instance extreme temperatures and chlorination of water.

Legionella are pathogens. In human they cause an acute bacterial pneumonia with facultative lethal course, which is generally known as “Legionnaire's disease”. This name is derived from the investigation of a striking accumulation of cases of pneumonia (189 cases with 29 deaths) among about 3000 delegates at the annual meeting of the “Pennsylvania Division of the American Legion” in July 1976. The investigation led to the isolation of a hitherto unknown bacterium, L. pneumophila (McDade et al., 1977. Legionnaire's disease: isolation of a bacterium and demonstration of its role in other respiratory disease. N. Engl. J. Med. 297 (22):1197-203), which was assigned to a new family, the Legionellaceae (Brenner, D. J. 1979, Speciation in Yersinia, p. 33-43. In: Carter, P. B., Lafleur, L. and Toma, S. (ed.), Contributions to microbiology and immunology, Vol. 5. Karger, Basel, Switzerland). Meanwhile, the so-called Pontiac Fever is known as another form of the disease caused by Legionella, which is characterized by flu-like symptoms and which has nothing to do with pneumonia. The reasons why patients develop one or the other disease form are not known.

The threat to life from the disease caused by Legionella as well as the ability of Legionella to survive under unfavorable living conditions for a long time show the need for a fast and reliable detection method.

Traditional detection of Legionella by means of cultivation is an extremely costly method which only leads to a result after several successive cultivation steps on different media within seven to 14 days.

Despite the great effort involved, cultivation has up to now been the method of choice for the detection of Legionella, since different alternative methods could not live up to the expectations placed in them.

For example, the analysis of suspicious samples on the basis of biochemical parameters, such as the determination of chinon profiles by HPLC or the fatty acid composition by GLC-MS (e.g. Ehret et al., 1987, Zentralbl. Bakteriol. Mikrobiol. Hyg. [A], 266 (1-2), 261-75) is not suitable for the routine diagnosis because of the very high expenditure of time and apparatus. Furthermore, the proper performance of these analyses calls for a high degree of qualification on the part of the personnel performing the analyses.

While the direct staining with fluorescent-labeled antibodies (DFA; direct fluorescent antibody staining) provides results within only a few hours, the method is neither sufficiently sensitive nor sufficiently specific. Only between 25% and 70% of the samples tested positive by cultivation were also positive by DFA (Zuravleff, J. L., V. L. Yu, J. L. Shonnard, 1983. Diagnosis of Legionnaires' disease and update of laboratory methods with new emphasis on isolation by culture. JAMA, Vol. 250, p. 1981-1985; Buesching, W. J., R. A. Brust, L. W. Ayers, 1983. Enhanced primary isolation of Legionella pneumophila from clinical specimens by low pH treatment. J. Clin. Microbiol., Vol. 17, p. 153-1155; Edelstein, P. H., 1987. The laboratory diagnosis of Legionnaires' disease. Sem. Respir. Infect., Vol. 2, p. 235-241). In addition, there are numerous species known which are also falsely stained by Legionella DFA conjugates, e.g. Pseudomonas fluorescens, P. aeruginosa and P. putida as well as different Bacteroides species. This inevitably leads to false positive results again and again. Furthermore, the immense variety of different Legionella serotypes is problematic when these test methods are used, as well as in all other methods based on binding of antibodies (e.g. RIA, ELISA, IFA). The large number of antisera necessary for the detection of all serotypes is hardly manageable, on the other hand if only a few antisera are used, the reliability of a negative test result is unacceptably low.

Numerous microbiological analyses are concerned with the investigation of Escherichia coli and coliform bacteria as so-called marker organisms. While, for example, in the testing of foodstuffs, drinking and surface water E. coli indicates a potential health risk as a so-called index organism, the coliform bacteria are regarded as indicators of generally inadequate hygiene. The testing of microbiological samples for index and indicator organisms allows to dispense with elaborate testing of the same samples for a variety of pathogens, since the presence of these bacteria is generally an indication of faecal contamination. Thus, the possible presence of other pathogens is very likely.

The coliform bacteria are an extremely heterogenous group of bacteria. The group of coliforms includes the genera Escherichia, Enterobacter, Klebsiella and Citrobacter. Whether bacteria belong to this group or not is thus not defined by taxonomic characteristics, but by the behavior of bacteria in the respective detection methods. To this extent all Gram-negative, aerobic, facultatively anaerobic, rod-like bacteria which are able to ferment lactose with the production of gas and acid within 48 hours at temperatures 30° C. and 37° C. are assigned to the coliforms. Coliforms which are able to ferment lactose at higher temperatures, namely at 44° C. to 45.5° C., are also called faecal coliforms, thermotrophic coliforms or presumptive E. coli.

While the sense of detecting coliforms has in the meantime become quite controversial (Means, E. G., Olson, B. H., 1981. Coliforms inhibition by bacteriocin-like substances in drinking water distribution systems. Appl. Environ. Microbiol., Vol. 42, p. 506-512; Burlingame, G. A.; McElhaney, J.; Pipes, W. O., 1984. Bacterial interference with coliform colony sheen production on membrane filters. Appl. Environ. Microbiol., Vol. 47, p. 56-60; Schmidt-Lorenz et al., 1988, Kritische Überlegungen zum Aussagewert von E. coli, Coliformen und Enterobacteriaceen in Lebensmitteln, Arch. Lebensmittelhyg. 39, 3-15.), there is no doubt about the value of the detection of E. coli as a marker organism.

In addition, E. coli serves not only as an index bacterium in microbiological analyses, but rather a number of pathogenic strains of this organism is known. These enterovirulent strains are divided into different subgroups (enterotoxin-producing, entero-pathogens, entero-hemorrhagic, entero-invasive, entero-adherent E. coli). All bacteria of these subgroups cause diarrhea diseases of different degrees of severity, right up to life-threatening ones.

Generally, the detection of E. coli and coliforms is carried out by cultivation, which, after several successive cultivation steps on different media produces a result within two to four days. As an alternative cultivation method, the cultivation on Fluorocult LMX-broth provides a result after only 30 hours. Also the membrane filter method for the detection of E. coli (the detection of coliforms is not possible with this method), still needs 22 to 32 hours until a result is obtained. But here not infrequently false-positive results are obtained, because especially in the case of fresh meat Indol-positive Klebsiella oxytoca and Providencia species are not infrequently found.

The so-called faecal streptococci are regarded as further indicators of faecal contamination of drinking and surface water. As in the case of the coliforms, they are also an inhomogeneous group. Faecal streptococci are assigned phylogenetically to the genera Streptococcus and Enterococcus. They are Gram-positive bacteria which typically produce diplococcae or short chains and are commonly found in the intestinal tract of warm-blooded animals.

The 2001 version of the German Drinking Water and Water for Food Factories Ordinance (Deutsche Verordnung für Trinkwasser und Wasser für Lebensmittelbetriebe) lays down limit values for faecal streptococci. No faecal streptococci may be traceable in 100 ml drinking water, otherwise the tested water is no longer of drinking water quality.

The detection methods recommended in the Drinking Water Ordinance are based on the direct cultivation of the water sample or an membrane filtration and subsequent introduction of the filter in 50 ml azide-glucose-broth. The cultivation should be carried out for at least 24 hours, in the case of a negative result for 48 hours at 36° C. If after 48 hours clouding or sedimentation of the broth is still not detectable, the absence of faecal streptococci in the tested sample is deemed to have been proven. In the case of clouding or sedimentation, streaking of the culture on enterococci selective agar according to Slanetz-Barthley and re-incubation at 36° C. for 24 hours takes place. If reddish-brown or pink colonies form, these will be examined in more detail. After transfer to a suitable liquid medium and cultivation for 24 hours at 36° C., faecal streptococci are deemed to have been detected when propagation in nutrient broth at a pH of 9.6 takes place and the propagation in 6.5% NaCl broth is possible as well as in the case of esculin degradation. Esculin degradation is checked by the addition of freshly prepared 7% aqueous solution of iron(II) chloride to aesculin broth. In the case of degradation a brownish-black color develops. Frequently, a Gram stain for differentiating bacteria from Gram-negative cocci is additionally carried out as well as a catalase test for differentiating from staphylococci. Faecal streptococci react Gram-positive and catalase-negative. The traditional detection procedure is thus shown to be tedious (48-100 hours) and, in suspected cases, an extremely elaborate method.

As a logical consequence of the difficulties presented by the above-mentioned methods for the detection of Legionella, E. coli and coliforms as well as faecal streptococci, detection methods on the basis of nucleic acids would be useful.

### SUMMARY OF THE INVENTION

Some embodiments relate to isolated oligonucleotides. The isolated oligonucleotides can be, for example, (i) an oligonucleotide with a nucleotide sequence of any of SEQ ID NOs. 1-47; (ii) an oligonucleotide which is at least 80%, 90%, 92%, 94%, or 96% identical to an oligonucleotide according to (i), and which render possible a specific hybridization with nucleic acid sequences of bacterial cells relevant to drinking water; (iii) an oligonucleotide, which differs from the oligonucleotides according to (i) by a deletion and/or addition, and renders possible a specific hybridization with a nucleic acid sequence of a bacterial cell relevant to drinking water; and (iv) an oligonucleotide hybridizing with a sequence complementary to an oligonucleotide according to i), ii) or iii) under stringent conditions.

Further embodiments relate to methods for detecting bacteria relevant to drinking water in a sample. The methods can include, for example, the steps of cultivating the bacteria relevant to drinking water present in the sample; fixing the bacteria relevant to drinking water present in the sample; incubating the fixed bacteria with at least one oligonucleotide according to claim 1 in order to achieve hybridization; removing non-hybridized oligonucleotides; detecting and visualizing the bacterial cells relevant to drinking water with the hybridized oligonucleotides. The methods can further include quantifying the bacterial cells relevant to drinking water with the hybridized oligonucleotides. The oligonucleotide can be linked to a detectable marker, including for example, a fluorescent marker, a chemoluminescence marker, a radioactive marker, an enzymatically active group, a haptene, and a nucleic acid detectable by hybridization. In any of the methods, the sample can be, for example, a drinking water sample or surface water sample. In the methods the detection can be performed by, for example, an optical microscope, epifluorescence microscope, chemoluminometer, fluorometer or flow cytometer. The bacteria relevant to drinking water can be for example, a bacteria of the genus Legionella, for example of the species L. pneumophila; a faecal streptococci; or a coliform bacteria, for example, of the species E. coli.

Also, embodiments relate to the methods as described herein used for the simultaneous specific detection of bacteria of the genus Legionella and the species L. pneumophila, wherein the oligonucleotide an oligonucleotide having the sequence of any of SEQ ID NOs:1-7, for example.

Other embodiments relate to the methods as described herein used for the specific detection of faecal streptococci, wherein the oligonucleotide is for example, an oligonucleotide with the sequence of any of SEQ ID NOs: 8-28. Still further embodiments relate to the described methods used for the simultaneous specific detection of coliform bacteria, including bacteria of the species Escherichia coli, wherein the oligonucleotide an oligonucleotide having the sequence of any of SEQ ID NOs: 29-47.

Some embodiments relate to methods for the detection of bacteria relevant to drinking water in a sample using, for example, (i) an oligonucleotide with a nucleotide sequence of any of SEQ ID NOs. 1-47; (ii) an oligonucleotide which is at least 80%, 90%, 92%, 94%, or 96% identical to an oligonucleotide according to (i), and which render possible a specific hybridization with nucleic acid sequences of bacterial cells relevant to drinking water; (iii) an oligonucleotide, which differs from the oligonucleotides according to (i) by a deletion and/or addition, and renders possible a specific hybridization with a nucleic acid sequence of a bacterial cell relevant to drinking water; and (iv) an oligonucleotide hybridizing with a sequence complementary to an oligonucleotide according to i), ii) or iii) under stringent conditions. The oligonucleotide can be used for the simultaneous specific detection of bacteria of the genus Legionella, including for example, the species L. pneumophila. For example, the oligonucleotide can have sequence of any of SEQ ID NOs: 1-7. Also, the oligonucleotides can be used for the simultaneous specific detection of bacteria of faecal streptococci. For example, the oligonucleotide can have a sequence of any of SEQ ID NOs: 8-28. Furthermore, the oligonucleotides can be used for the simultaneous specific detection of coliform bacteria, including those of the species Escherichia coli. For example, the oligonucleotide can have the sequence of any of SEQ ID NOs:29-47.

Other embodiments relate to kits for performing any of the described methods. The kits can include, for example, at least one of the following, (i) an oligonucleotide with a nucleotide sequence of any of SEQ ID NOs. 1-47; (ii) an oligonucleotide which is at least 80%, 90%, 92%, 94%, or 96% identical to an oligonucleotide according to (i), and which render possible a specific hybridization with nucleic acid sequences of bacterial cells relevant to drinking water; (iii) an oligonucleotide, which differs from the oligonucleotides according to (i) by a deletion and/or addition, and renders possible a specific hybridization with a nucleic acid sequence of a bacterial cell relevant to drinking water; and (iv) an oligonucleotide hybridizing with a sequence complementary to an oligonucleotide according to i), ii) or iii) under stringent conditions. The kits can include, for example, at least one oligonucleotide in a hybridization solution. Also, the kits can include a washing solution and/or one or more fixation solutions.

### DETAILED DESCRIPTION OF THE PREFERRED EMBODIMENT

In PCR, polymerase chain reaction, a characteristic piece of the respective bacterial genome is amplified with specific primers. If a primer finds its target site, a million-fold amplification of a piece of the inherited material occurs. Upon the following analysis, for example by an agarose gel separating DNA fragments, a qualitative evaluation can take place. In the most simple case this leads to the conclusion that target sites for the primers used were present in the tested sample. Further conclusions are not possible; these target sites can originate from both a living bacterium and a dead bacterium or from naked DNA. Differentiation is not possible with this method. This is particularly problematic when testing samples for ubiquitous germs such as E. coli and coliforms. This often leads to false positive results, since the PCR reaction is positive also in the presence of a dead bacterium or naked DNA. A further refinement of this technique is the quantitative PCR, which tries to establish a correlation between the amount of bacteria present and the amount of amplified DNA. Advantages of PCR are its high specificity, its ease of application and its low expenditure of time. Its main disadvantages are its high susceptibility to contamination and therefore false positive results, as well as the aforementioned lack of possibility to discriminate between living and dead cells or naked DNA, respectively.

A unique approach to combine the specificity of molecular biological methods such as PCR with the possibility of the visualization of bacteria, which is facilitated by the antibody methods, is the method of fluorescence in situ hybridization (FISH; Amann, R. I., W. Ludwig and K.-H. Schleifer, 1995. Phylogenetic identification and in situ detection of individual microbial cells without cultivation. Microbiol. Rev. 59, p. 143-169). Using this method bacteria species, genera or groups can be identified and visualized with high specificity.

The FISH technique is based on the fact that in bacteria cells there are certain molecules which have only been mutated to a small extent in the course of evolution because of their essential function. These are the 16S and the 23S ribosomal ribonucleic acid (rRNA). Both are parts of the ribosomes, the sites of protein biosynthesis, and can serve as specific markers on account of their ubiquitous distribution, their size and their structural and functional constancy (Woese, C. R., 1987. Bacterial evolution. Microbiol. Rev. 51, p. 221-271). Based on a comparative sequence analysis, phylogenetic relationships can be established based on these data alone. For this purpose, the sequence data have to be brought into an alignment. In the alignment, which is based on the knowledge about the secondary structure and tertiary structure of these macromolecules, the homologous positions of the ribosomal nucleic acids are brought into line with each other.

Based on these data, phylogenetic calculations can be made. The use of the most modern computer technology makes it possible to make even large-scale calculations fast and effectively, as well as to set up large databases which contain the alignment sequences of 16S rRNA and 23S rRNA. Because of the fast access to this data material, newly acquired sequences can be phylogenetically analyzed within a short time. These rRNA databases can be used to construct species-specific and genus-specific gene probes. Here all available rRNA sequences are compared with each other and probes are designed for specific sequence sites, which probes cover a specific species, genus or group of bacteria.

In the FISH (fluorescence in situ hybridization) technique, these gene probes, which are complementary to a certain region on the ribosomal target sequence, are brought into the cell. The gene probes are generally small, 16-20 bases long, single-stranded deoxyribonucleic acid pieces and are directed against a target region which is typical for a bacterial species or a bacterial group. If a fluorescence labeled gene probe finds its target sequence in a bacterial cell, it binds to it and the cells can be detected in the fluorescence microscope because of their fluorescence.

The FISH analysis is always performed on a slide, because for the evaluation the bacteria are visualized by irradiation with a high-energy light. But herein lies one of the disadvantages of the classical FISH analysis: because naturally only relatively small volumina can be analyzed on the slide, the sensitivity of the method may be unsatisfactory and not sufficient for a reliable analysis. The present invention thus combines the advantages of the classical FISH analysis with those of cultivation. A comparatively short cultivation step ensures that the bacteria to be detected are present in sufficient number before the bacteria are detected using specific FISH.

Realization of the methods described in the present application for the simultaneous specific detection of bacteria of the genus Legionella as well as the species L. pneumophila or for the specific detection of faecal streptococci or for the simultaneous specific detection of coliform bacteria and bacteria of the species E. coli comprises the following steps:
cultivating the bacteria present in the sample to be tested
fixing the bacteria present in the sample
incubating the fixed bacteria with nucleic acid probe molecules, in order to achieve hybridization,
removing or washing off the non-hybridized nucleic acid probe molecules and
detecting the bacteria hybridized with the nucleic acid probe molecules.

Within the scope of the present invention “cultivating” is understood to mean the propagation of the bacteria present in the sample in a suitable cultivation medium. Methods suitable for this purpose are well known to those of skill in the art.

Within the scope of the present invention “fixing” of the bacteria is understood to mean a treatment with which the bacterial envelope is made permeable for nucleic acid probes. For fixation, usually ethanol is usually used. If the cell wall cannot be penetrated by the nucleic acid probes using these techniques, the skilled artisan will know a sufficient number of other techniques which lead to the same result. These include, for example, methanol, mixtures of alcohols, low percentage paraformaldehyde solution or a diluted formaldehyde solution, enzymatic treatments or the like.

Within the scope of the present invention the fixed bacteria are incubated with fluorescence labeled nucleic acid probes for the “hybridization”. These nucleic acid probes, which consist of an oligonucleotide and a marker linked thereto can then penetrate the cell wall and bind to the target sequence corresponding to the nucleic acid probe in the cell. Binding is to be understood as formation of hydrogen bonds between complementary nucleic acid pieces.

The nucleic acid probe here can be complementary to a chromosomal or episomal DNA, but also to an mRNA or rRNA of the microorganism to be detected. It is advantageous to select a nucleic acid probe which is complementary to a region present in copies of more than 1 in the microorganism to be detected. The sequence to be detected is preferably present in 500-100,000 copies per cell, especially preferred 1,000-50,000 copies. For this reason the rRNA is preferably used as a target site, since the ribosomes as sites of protein biosynthesis are present many thousand-fold in each active cell.

The nucleic acid probe within the meaning of the invention may be a DNA or RNA probe comprising usually between 12 and 1,000 nucleotides, preferably between 12 and 500, more preferably between 12 and 200, especially preferably between 12 and 50 and between 15 and 40, and most preferably between 17 and 25 nucleotides. The selection of the nucleic acid probes is done according to criteria of whether a complementary sequence is present in the microorganism to be detected. By selecting a defined sequence, a bacterial species, a bacterial genus or an entire bacterial group may be detected. In a probe consisting of 15 nucleotides, the sequences should be 100% complementary. In oligonucleotides of more than 15 nucleotides, one or more mismatches are allowed.

By complying with stringent hybridization conditions it is guaranteed that the nucleic acid probe molecule indeed hybridizes with the target sequence. As explained in more detail below, stringent conditions within the meaning of the invention are e.g. 20-80% formamide in the hybridization buffer.

Besides this, stringent conditions can of course be found in the literature and standard works (such as, for instance, Manual of Sambrook et al. (1989) Cold Spring Harbor Laboratory Press, Cold Spring Harbor, N.Y.). Generally, “specific hybridizing” means that a molecule preferentially binds to a certain nucleotide sequence under stringent conditions, if this sequence is in a complex mixture of (e.g. total) DNA or RNA. The term “stringent conditions” stands for conditions under which a probe preferentially hybridizes to its target sequence and to a significantly lesser extent or not at all to other sequences. Stringent conditions are partly sequence-dependent and will vary under different conditions. Longer sequences specifically hybridize at higher temperatures. Generally, the stringent conditions are selected in such a way that the temperature is about 5° C. below thermal melting point (Tₘ) for the specific sequence at a defined ionic strength and a defined pH. The Tₘ is the temperature (at defined ionic strength, pH and nucleic acid concentration), at which 50% of the probe molecules complementary to the target sequence hybridize to the target sequence in a state of equilibrium. (As the target sequences are usually in excess, 50% of the probes are occupied in the state of equilibrium. Typically, stringent conditions are those at which the salt concentration is at least about 0.01 to 1.0 M sodium ion concentration (or another salt) at a pH between 7.0 and 8.3 and the temperature is at least about 30° C. for short probes (meaning, for instance, 10-50 nucleotides). Additionally, stringent conditions as mentioned above can be achieved by the addition of destabilizing agents, as for example formamide.

Within the scope of the method of the present invention the nucleic acid probe molecules of the present invention have the following lengths and sequences (all sequences are in 5′-3′ direction).

Method for the simultaneous specific detection of bacteria of the genus Legionella and the species L. pneumophila.

| | |
|---|---|
| 5′- cac tac cct ctc cca tac | (SEQ ID NO:1) |
| 5′- cac tac cct ctc cta tac | (SEQ ID NO:2) |
| 5′- c cac cac cct ctc cca tac | (SEQ ID NO:3) |
| 5′- c cac ttc cct ctc cca tac | (SEQ ID NO:4) |
| 5′- c cac tac cct ctc ccg tac | (SEQ ID NO:5) |
| 5′- c cac tac cct cta cca tac | (SEQ ID NO:6) |
| 5′- t atc tga ccg tcc cag gtt a | (SEQ ID NO:7) |

Method for the specific detection of faecal streptococci:

| | |
|---|---|
| 5′- ccc tct gat ggg tag gtt | (SEQ ID NO:8) |
| 5′- ccc tct gat ggg cag gtt | (SEQ ID NO:9) |
| 5′- tag gtg ttg tta gca ttt cg | (SEQ ID NO:10) |
| 5′- cac tcc tct ttt tcc ggt | (SEQ ID NO:11) |
| 5′- c cac ttc tct ttt tcc ggt | (SEQ ID NO:12) |
| 5′- c cac tct tct ttt tcc ggt | (SEQ ID NO:13) |
| 5′- c cac tct tct ttt ccc ggt | (SEQ ID NO:14) |
| 5′- cac aca atc gta aca tcc ta | (SEQ ID NO:15) |
| 5′- agg gat gaa ctt tcc act c | (SEQ ID NO:16) |
| 5′- cca ctc att ttc ttc cgg | (SEQ ID NO:17) |
| 5′- ccc ccg ctt gag ggc agg | (SEQ ID NO:18) |
| 5′- cct ctt ttc ccg gtg gag | (SEQ ID NO:19) |
| 5′- cct ctt ttt ccg gtg gag c | (SEQ ID NO:20) |
| 5′- cac tcc tct ttt cca atg a | (SEQ ID NO:21) |
| 5′- cac tcc tct tac ttg gtg | (SEQ ID NO:22) |
| 5′- tag gtg cca gtc aaa ttt tg | (SEQ ID NO:23) |
| 5′- ccc ctt ctg atg ggc agg | (SEQ ID NO:24) |
| 5′- ccc cct ctg atg ggc agg | (SEQ ID NO:25) |
| 5′- cga ctt cgc aac tcg ttg | (SEQ ID NO:26) |
| 5′- cga ctt cgc gac tcg ttg | (SEQ ID NO:27) |
| 5′- cga gtt cgc aac tcg ttg | (SEQ ID NO:28) |

Method for the simultaneous specific detection of coliform bacteria and bacteria of the species Escherichia coli:

| | |
|---|---|
| 5′- gac ccc ctt gcc gaa a | (SEQ ID NO:29) |
| 5′- atg acc ccc tag ccg aaa | (SEQ ID NO:30) |
| 5′- ggc aca acc tcc aag tcg ac | (SEQ ID NO:31) |
| 5′- gga caa cca gcc tac atg ct | (SEQ ID NO:32) |
| 5′- aca aga ctc cag cct gcc | (SEQ ID NO:33) |
| 5′- cag gcg gtc tat tta acg cgt t | (SEQ ID NO:34) |
| 5′- ggc aca acc tcc aaa tcg ac | (SEQ ID NO:35) |
| 5′- ggc cac aac ctc caa gta ga | (SEQ ID NO:36) |
| 5′- acc aca ctc cag cct gcc | (SEQ ID NO:37) |
| 5′- aca aga ctc tag cct gcc | (SEQ ID NO:38) |
| 5′- ggc ggt cga ttt aac gcg tt | (SEQ ID NO:39) |
| 5′- ggc ggt cta ctt aac gcg tt | (SEQ ID NO:40) |
| 5′- ggc ggt cta ttt aat gcg tt | (SEQ ID NO:41) |
| 5′- agc tcc gga agc cac tcc tca | (SEQ ID NO:42) |
| 5′- gga aca acc tcc aag tcg | (SEQ ID NO:43) |
| 5′- gcc aca acc tcc aag tag | (SEQ ID NO:44) |
| 5′- atg gcc ccc tag ccg aaa | (SEQ ID NO:45) |
| 5′- g atg acc ccc tag ccg aaa | (SEQ ID NO:46) |
| 5′- aac ctt gcg gcc gta ctc cc | (SEQ ID NO:47) |

A further object of the invention are modifications of the above oligonucleotide sequences, demonstrating specific hybridization with target nucleic acid sequences of the respective bacterium despite variations in sequence and/or length, and which are therefore suitable for use in a method according to the invention. These especially include:
a) nucleic acid molecules (i) being identical to one of the above oligonucleotide sequences (SEQ ID No. 1 to SEQ ID No. 47) to at least 80%, 84%, 87% and preferably to at least 90%, 92% and particularly preferred to at least 94%, 96%, 98% of the bases (wherein the sequence region of the nucleic acid molecule is to be considered which corresponds to the sequence region of one of the above oligonucleotides (SEQ ID No. 1 to SEQ ID No. 47) and not the entire sequence of a nucleic acid molecule, which possibly may be extended by one or multiple bases compared to the above-mentioned oligonucleotides (SEQ ID No. 1 to SEQ ID No. 47), or (ii) differs from the above oligonucleotide sequences (SEQ ID No. 1 to SEQ ID No. 47) by one or more deletions and/or additions and which render possible a specific hybridization with nucleic acid sequences of bacteria of the genus Legionella and the species L. pneumophila, of faecal streptococci or of coliform bacteria and bacteria of the species E. coli. In this context “specific hybridization” means that under the hybridization conditions described here or those known to the person skilled in the art in relation to in situ hybridization techniques, only the ribosomal RNA of the target organisms binds to the oligonucleotide, but not the rRNA of non-target organisms.
b) Nucleic acid molecules which are complementary to the nucleic acid molecules mentioned in a) or to one of the probes SEQ ID No. 1 to SEQ ID No. 47 or which specifically hybridize with these under stringent conditions.
c) Nucleic acid molecules comprising an oligonucleotide sequence of SEQ ID No. 1 to SEQ ID No. 47 or the sequence of a nucleic acid molecule according to a) or b) and having at least one further nucleotide in addition to the mentioned sequences or their modifications according to a) or b) and allowing specific hybridization with nucleic acid sequences of target organisms.

The degree of sequence identity of a nucleic acid molecule to the probes SEQ ID No. 1 to SEQ ID No. 47 can be determined using the usual algorithms. In this respect, for example, the program for determining the sequence identity available under hypertext transfer protocol accessible on the world wide web at “ncbi.nlm.nih.gov/BLAST” (http://www.ncbi.nlm.nih.gov/BLAST) (on this page for example the link “Standard nucleotide-nucleotide BLAST [blastn]”) is suitable.

“Hybridization” within the scope of this invention can be synonymous with “complementary”. Within the scope of this invention also those oligonucleotides are comprised which hybridize with the (theoretical) counterstrand of an oligonucleotide according to the present invention, including the modifications according to the invention of SEQ ID No. 1 to 47.

The nucleic acid probe molecules according to the invention may be used within the scope of the detection method with various hybridization solutions. Various organic solvents may be used in concentrations of 0-80%. By keeping stringent hybridization conditions, it is guaranteed that the nucleic acid probe molecule indeed hybridizes to the target sequence. Moderate conditions within the meaning of the invention are e.g. 0% formamide in a hybridization buffer as described below. Stringent conditions within the meaning of the invention are for example 20-80% formamide in the hybridization buffer.

Within the scope of the method according to the invention for simultaneous specific detection of bacteria of the genus Legionella and the species L. pneumophila a typical hybridization solution contains 0%-80% formamide, preferably 20%-60% formamide, especially preferred 35% formamide. In addition, it has a salt concentration of 0.1 mol/l-1.5 mol/l, preferably of 0.5 mol/1-1.0 mol/l, more preferred of 0.7 mol/l-0.9 mol/l and especially preferred of 0.9 mol/l, the salt preferably being sodium chloride. Further, the hybridization solution usually comprises a detergent, such as for instance sodium dodecyl sulfate (SDS) in a concentration of 0.001%-0.2%, preferably in a concentration of 0.005-0.05%, more preferred of 0.01-0.03%, especially preferred in a concentration of 0.01%. For buffering of the hybridization solution, various compounds such as Tris-HCl, sodium citrate, PIPES or HEPES may be used, which are usually used in concentrations of 0.01-0.1 mol/l, preferably of 0.01 to 0.08 mol/l, in a pH range of 6.0-9.0, preferably 7.0 to 8.0. The particularly preferred inventive embodiment of the hybridization solution contains 0.02 mol/l Tris-HCl, pH 8.0.

Within the scope of the method according to the invention for the specific detection of faecal streptococci, a typical hybridization solution contains 0%-80% formamide, preferably

20%-60% formamide, particularly preferred 35% formamide. In addition it has a salt concentration of 0.1 mol/l-1.5 mol/l, preferably of 0.5 mol/l to 1.0 mol/l, preferably of 0.7 mol/l to 0.9 mol/l, particularly preferred of 0.9 mol/l, the salt preferably being sodium chloride. Further, the hybridization solution usually comprises a detergent, such as for example sodium dodecyl sulfate (SDS), in a concentration of 0.001%-0.2%, preferably in a concentration of 0.005-0.05%, more preferably 0.01-0.03%, especially preferred in a concentration of 0.01%. For buffering of the hybridization solution, various compounds such as Tris-HCl, sodium citrate, PIPES or HEPES may be used, which are usually used in concentrations of 0.01-0.1 mol/l, preferably of 0.01 to 0.08 mol/l, in a pH range of 6.0-9.0, preferably 7.0 to 8.0. The particularly preferred inventive embodiment of the hybridization solution contains 0.02 mol/l Tris-HCl, pH 8.0.

Within the scope of the method of the present invention for the simultaneous specific detection of coliform bacteria and the species E. coli, a typical hybridization solution contains 0%-80% formamide, preferably 20%-60% formamide, especially preferred 50% formamide. In addition it has a salt concentration of 0.1 mol/l-1.5 mol/l, preferably of 0.7 mol/1-0.9 mol/l, especially preferred of 0.9 mol/l, the salt preferably being sodium chloride. Further, the hybridization solution usually comprises a detergent such as for example sodium dodecyl sulfate (SDS), in a concentration of 0.001-0.2%, preferably in a concentration of 0.005-0.05%, more preferably 0.01-0.03%, especially preferred in a concentration of 0.01%. For buffering of the hybridization solution, various compounds, such as Tris-HCl, sodium citrate, PIPES or HEPES may be used, which are usually used in concentrations of 0.01-0.1 mol/l, preferably of 0.01 to 0.08 mol/l, in a pH range of 6.0-9.0, preferably 7.0 to 8.0. The particularly preferred inventive embodiment of the hybridization solutions contains 0.02 mol/l Tris-HCl, pH 8.0.

It shall be understood that the skilled artisan can choose the given concentrations of the constituents of the hybridization buffer in such a way that the desired stringency of the hybridization reaction is achieved. Especially preferred embodiments reflect stringent to particularly stringent hybridization conditions. Using these stringent conditions one or skill in the art can determine whether a particular nucleic acid molecule enables the specific detection of nucleic acid sequences of target organisms and may thus be reliably used within the scope of the invention.

The concentration of the probe may vary greatly, depending on the label and number of target structures to be expected. In order to allow rapid and efficient hybridization, the probe amount should exceed the number of the target structures by several orders of magnitude. However, it has to be noted that in fluorescence in situ hybridization (FISH) too high levels of fluorescence labelled hybridization probe results in increased background fluorescence. The amount of probe should therefore be between 0.5 ng/μl and 500 ng/μl, preferably between 1.0 ng/μl and 100 ng/μl, and especially preferred at 1.0-50 ng/μl.

Within the scope of the method of the present invention the preferred concentration is 1-10 ng for each nucleic acid molecule used per μl hybridization solution. The volume of hybridization solution used should be between 8 μl and 100 ml, in an especially preferred embodiment of the method of the present invention it is 40 μl.

The hybridization usually lasts between 10 minutes and 12 hours. Preferably, the hybridization lasts for about 1.5 hours. The hybridization temperature is preferably between 44° C. and 48° C., especially preferred 46° C., wherein the parameter of the hybridization temperature as well as the concentration of salts and detergents in the hybridization solution may be optimized depending on the nucleic acid probes, especially their length and the degree to which they are complementary to the target sequence in the cell to be detected. The skilled artisan is familiar with the appropriate calculations.

After hybridization the non-hybridized and excess nucleic acid probe molecules should be removed or washed off, which is usually achieved by a conventional washing solution. This washing solution may, if desired, contain 0.001-0.1% of a detergent such as SDS, a concentration of 0.01% being preferred, as well as Tris-HCl in a concentration of 0.001-0.1 mol/l, preferably 0.01-0.05 mol/l, especially preferred 0.02 mol/l, wherein the pH value of Tris-HCl is within the range of 6.0 to 9.0, preferably of 7.0 to 8.0, especially preferred 8.0. A detergent may be contained, although this is not absolutely necessary. Furthermore, the washing solution usually contains NaCl, wherein the concentration is 0.003 mol/l to 0.9 mol/l, preferably 0.01 mol/l to 0.9 mol/l, depending on the stringency required. An NaCl concentration of 0.07 mol/l (method for the simultaneous specific detection of bacteria of the genus Legionella and the species Lpneumophila) or 0.07 mol/l (method for the specific detection of faecal streptococci) or 0.018 mol/l (method for the simultaneous specific detection of coliform bacteria and bacteria of the species E. coli) is especially preferred. Moreover, the washing solution may contain EDTA in a concentration of up to 0.01 mol/l, wherein the concentration is preferably 0.005 mol/l. The washing solution may further contain suitable amounts of preservatives known to the skilled artisan.

Generally, buffer solutions are used in the washing step, which can in principle be very similar to the hybridization buffer (buffered sodium chloride solution), except that the washing step is performed in a buffer with a lower salt concentration or at a higher temperature.

For theoretical estimation of the hybridization conditions, the following formula may be used:
Td=81.5+16.6 lg[Na⁺]+0.4×(% GC)−820/n−0.5×(% FA)
Td=dissociation temperature in ° C.
[Na⁺]=molarity of the sodium ions
% GC=percentage of guanine and cytosine nucleotides relative to the number of total bases
n=hybrid length
% FA=percentage of formamide

Using this formula, the formamide content (which should be as low as possible due to its toxicity) of the washing buffer may for example be replaced by a correspondingly lower sodium chloride content. However, the person skilled in the art knows from the extensive literature concerning in situ hybridization methods the fact that, and in which way, the mentioned contents can be varied. Concerning the stringency of the hybridization conditions, the same applies as outlined above for the hybridization buffer.

The “washing off” of the non-bound nucleic acid probe molecules is usually performed at a temperature in the range of 44° C. to 52° C., preferably from 44° C. to 50° C. and especially preferred at 46° C. for 10-40 minutes, preferably for 15 minutes.

In an alternative embodiment of the method according to the invention, the nucleic acid probe molecules according to the invention are used in the so-called Fast-FISH method for the specific detection of the mentioned target organisms. The Fast-FISH method is known to the skilled artisan and is, for example, described in German patent application DE 199 36 875.9 and in the international application WO 99/18234. Reference is herewith expressly made to the disclosure contained in these documents regarding the performance of the detection methods described therein.

The specifically hybridized nucleic acid probe molecules can then be detected in the respective cells, provided that the nucleic acid probe molecule is detectable, e.g. by linking the probe molecule to a marker by covalent binding. As detectable markers, for example, fluorescent groups, such as for example CY2 (available from Amersham Life Sciences, Inc., Arlington Heights, USA), CY3 (also available from Amersham Life Sciences), CY5 (also obtainable from Amersham Life Sciences), FITC (Molecular Probes Inc., Eugene, USA), FLUOS (available from Roche Diagnostics GmbH, Mannheim, Germany), TRITC (available from Molecular Probes Inc., Eugene, USA), 6-FAM or FLUOS-PRIME are used, which are well known to the person skilled in the art. Also chemical markers, radioactive markers or enzymatic markers, such as horseradish peroxidase, acid phosphatase, alkaline phosphatase, peroxidase may be used. For each of these enzymes a number of chromogens is known which may be converted instead of the natural substrate and may be transformed to either coloured or fluorescent products. Examples of such chromogens are listed in the following table:

TABLE

| Enzyme | Chromogen |
|---|---|
| 1. Alkaline phosphatase | 4-methylumbelliferyl phosphate (*), bis(4- |
| and acid phosphatase | methylumbelliferyl phosphate, (*) 3-O-methylfluorescein, |
| | flavone-3-diphosphate triammonium salt (*), p- |
| | nitrophenylphosphate disodium salt |
| 2. Peroxidase | tyramine hydrochloride (*), 3-(p-hydroxyphenyl)- |
| | propionate (*), p-hydroxyphenethyl alcohol (*), 2,2′-azino- |
| | di-3-ethylbenzothiazoline sulfonic acid (ABTS), ortho- |
| | phenylendiamine dihydrochloride, o-dianisidine, 5- |
| | aminosalicylic acid, p-ucresol (*), 3,3′-dimethyloxy |
| | benzidine, 3-methyl-2-benzothiazoline hydrazone, |
| | tetramethylbenzidine |
| 3. Horseradish peroxidase | H₂O₂ + diammonium benzidine |
| | H₂O₂ + tetramethylbenzidine |
| 4. β-D-galactosidase | o-nitrophenyl-β-D-galactopyranoside, |
| | 4-methylumbelliferyl-β-D-galactoside |
| 5. Glucose oxidase | ABTS, glucose and thiazolyl blue |

* fluorescence

Finally, it is possible to generate the nucleic acid probe molecules in such a way that another nucleic acid sequence suitable for hybridization is present at their 5′ or 3′ ends. This nucleic acid sequence in turn comprises about 15 to 1,000, preferably 15-50 nucleotides. This second nucleic acid region may in turn be detected by a nucleic acid probe molecule, which is detectable by one of the above-mentioned agents.

Another possibility is the coupling of the detectable nucleic acid probe molecules to a haptene which may subsequently be brought into contact with a haptene-recognising antibody. Digoxigenin may be mentioned as an example of such a haptene. Other examples in addition to those mentioned are well known to the person of skill in the art.

The final evaluation depends on the kind of labelling of the probe used and is possible with an optical microscope, epifluorescence microscope, chemoluminometer, fluorometer, etc.

An important advantage of the methods described in this application for the simultaneous specific detection of bacteria of the genus Legionella and the species L. pneumophila or for the specific detection of faecal streptococci or methods for the simultaneous specific detection of coliform bacteria and bacteria of the species E. coli compared to the detection methods described above is the speed. In comparison to conventional cultivation methods which need seven to 14 days for the detection of Legionella, 48 to 100 hours for the detection of faecal streptococci and 30 to 96 hours for the detection of coliform bacteria and E. coli, respectively, the results when the method according to the invention is used are obtained within 24-48 hours.

Another advantage is the simultaneous detection of bacteria of the genus Legionella and the species L. pneumophila. With the methods common up to now only bacteria of the species L. pneumophila could be detected more or less reliably. Epidemiological investigations however have shown that besides L. pneumophila also other species of the genus Legionella can cause the dangerous Legionnaires' Disease, for example Legionella micdadei. According to the information presently available, the detection of L. pneumophila alone can no longer be considered sufficient.

Another advantage is the possibility to discriminate between bacteria of the genus Legionella and those of the species L. pneumophila. This is possible easily and reliably by using differently labeled nucleic acid probe molecules.

Another advantage is the specificity of these methods. With the nucleic acid probe molecules used, not only specifically all species of the genus Legionella, but also the species L. pneumophila alone can be detected and visualized with high specificity. Equally reliably, all species of the heterogeneous groups of faecal streptococci and coliforms can be detected as well as all sub-groups of the species E. coli. By visualization of the bacteria a visual control may be performed at the same time. False-positive results are therefore ruled out.

A further advantage of the method according to the invention is its ease of use. For example, using this method, large amounts of samples can easily be tested for the presence of the mentioned bacteria.

The methods according to the invention may be used in various ways.

For example, environmental samples can be tested for the presence of Legionella. These samples may be collected for instance from water or from soil.

The method according to the invention can further be used to test medical samples. It is suitable for the analysis of samples obtained from sputum, broncho-alveolar lavage or endotrachial suction. It is further suitable for the analysis of tissue samples, e.g. biopsy material from the lung, tumor or inflamed tissue, from secretions such as sweat, saliva, semen and discharges from the nose, uretha or vagina as well as for urine and stool samples.

Another field of application for the present method is the analysis of waters, e.g. shower and bath waters or drinking water.

Another field of application of the method according to the invention is the control of foodstuffs. In preferred embodiments the food samples are obtained from milk or milk products (yogurt, cheese, sweet cheese, butter, buttermilk), drinking water, beverages (lemonades, beer, juices), bakery products or meat products.

A further field of application of the method according to the invention is the analysis of pharmaceutical and cosmetic products, e.g. ointments, creams, tinctures, juices, solutions, drops, etc.

Furthermore, according to the invention, three kits for performing the respective methods are provided. The hybridization arrangement contained in these kits is described for example in German patent application 100 61 655.0. Express reference is herewith made to the disclosure contained in this document with respect to the in situ hybridization arrangement.

Besides the described hybridization arrangement (referred to as VIT reactor), the most important component of the kits is the respective hybridization solution (referred to as VIT solution) with the nucleic acid probe molecules specific for the microorganisms to be detected, which are described above. Further contained are the respective hybridization buffer (Solution C) and a concentrate of the respective washing solution (Solution D). Also contained are optionally fixation solutions (Solution A and Solution B) as well as an embedding solution (finisher). Finishers are commercially available, they prevent, among other things, the rapid bleaching of fluorescent probes under the fluorescence microscope. Optionally, solutions for parallel carrying out of a positive control as well as of a negative control are contained.

The following example is intended to illustrate the invention without limiting it.

### EXAMPLE

Specific rapid detection of bacteria relevant to drinking water in a sample

A sample is cultivated for 20-44 hours in a suitable manner. Various suitable methods are well known to a person of skill in the art. To an aliquot of this culture the same volume of fixation solution (Solution A, 50% ethanol) is added.

For hybridization, a suitable aliquot of the fixed cells (preferably 40 μl) is applied onto a slide and dried (46° C., 30 min or until completely dry). Then the dried cells are completely dehydrated by adding another fixation solution (Solution B, ethanol absolute, preferably 40 μl). The slide is again dried (room temperature, 3 min or until completely dry).

Then the hybridization solution (VIT solution) containing the above described nucleic acid probe molecules specific for the microorganisms to be detected is applied to the fixed, dehydrated cells. The preferred volume is 40 μl. The slide is then incubated in a chamber humidified with hybridization buffer (Solution C, corresponding to the hybridization solution without probe molecules), preferably the VIT reactor (46° C., 90 min).

Then the slide is removed from the chamber, the chamber is filled with washing solution (Solution D, diluted 1:10 with distilled water) and the slide is incubated in the chamber (46° C., 15 min).

Then the chamber is filled with distilled water, the slide is briefly immersed and then air-dried in lateral position (46° C., 30 min or until completely dry).

Then the slide is embedded in a suitable medium (finisher).

Finally, the sample is analyzed with the help of a fluorescence microscope.

1. An isolated oligonucleotide, selected from the group consisting of:
i) an oligonucleotide with a nucleotide sequence of any of SEQ ID NOs. 1-47;
ii) an oligonucleotide which is at least 80% identical to the oligonucleotides according to (i), and which renders possible a specific hybridization with nucleic acid sequences of bacterial cells relevant to drinking water;
iii) an oligonucleotide, which differs from the oligonucleotides according to (i) by a deletion and/or addition, and renders possible a specific hybridization with a nucleic acid sequence of a bacterial cell relevant to drinking water; and
iv) an oligonucleotide hybridizing with a sequence complementary to an oligonucleotide according to i), ii) or iii) under stringent conditions.

2. A method for detecting bacteria relevant to drinking water in a sample, comprising the steps of:
a) cultivating the bacteria relevant to drinking water present in the sample;
b) fixing the bacteria relevant to drinking water present in the sample;
c) incubating the fixed bacteria with at least one oligonucleotide according to claim 1 in order to achieve hybridization;
d) removing non-hybridized oligonucleotides;
e) detecting and visualizing the bacterial cells relevant to drinking water with the hybridized oligonucleotides.

3. The method according to claim 2, further comprising quantifying the bacterial cells relevant to drinking water with the hybridized oligonucleotides.

4. The method according to claim 2, wherein the detection is performed by an optical microscope, epifluorescence microscope, chemoluminometer, fluorometer or flow cytometer.

5. The method according to claim 2, wherein the sample is a drinking water sample or surface water sample.

6. The method according to claim 2, wherein the oligonucleotide is linked to a detectable marker, selected from the group consisting of:
a) a fluorescent marker,
b) a chemoluminescence marker,
c) a radioactive marker,
d) an enzymatically active group,
e) a haptene, and
f) a nucleic acid detectable by hybridization.

7. Method according to claim 6, wherein the sample is a drinking water sample or surface water sample.

8. The method according to claim 6, wherein the detection is performed by an optical microscope, epifluorescence microscope, chemoluminometer, fluorometer or flow cytometer.

9. The method according to claim 2, wherein the bacteria relevant to drinking water is a bacteria of the genus Legionella, faecal streptococci, or coliform bacteria.

10. The method of claim 9, wherein the Legionella species is L. pneumophila, or the coliform bacteria species is E. coli.

11. The method according to claim 2 for the simultaneous specific detection of bacteria of the genus Legionella, wherein the oligonucleotide is selected from the group consisting of

| | |
|---|---|
| 5′- cac tac cct ctc cca tac, | (SEQ ID NO:1) |
| 5′- cac tac cct ctc cta tac, | (SEQ ID NO:2) |
| 5′- c cac cac cct ctc cca tac, | (SEQ ID NO:3) |
| 5′- c cac ttc cct ctc cca tac, | (SEQ ID NO:4) |
| 5′- c cac tac cct ctc ccg tac, | (SEQ ID NO:5) |
| 5′- c cac tac cct cta cca tac, | (SEQ ID NO:6) |
| and | |
| 5′- t atc tga ccg tcc cag gtt a. | (SEQ ID NO:7) |

12. The method according to claim 11, wherein the Legionella species is L. pneumophila.

13. The method according to claim 2 for the specific detection of faecal streptococci, wherein the oligonucleotide is selected from the group consisting of

| | |
|---|---|
| 5′- ccc tct gat ggg tag gtt, | (SEQ ID NO:8) |
| 5′- ccc tct gat ggg cag gtt, | (SEQ ID NO:9) |
| 5′- tag gtg ttg tta gca ttt cg, | (SEQ ID NO:10) |
| 5′- cac tcc tct ttt tcc ggt, | (SEQ ID NO:11) |
| 5′- c cac ttc tct ttt tcc ggt, | (SEQ ID NO:12) |
| 5′- c cac tct tct ttt tcc ggt, | (SEQ ID NO:13) |
| 5′- c cac tct tct ttt ccc ggt, | (SEQ ID NO:14) |
| 5′- cac aca atc gta aca tcc ta, | (SEQ ID NO:15) |
| 5′- agg gat gaa ctt tcc act c, | (SEQ ID NO:16) |
| 5′- cca ctc att ttc ttc cgg, | (SEQ ID NO:17) |
| 5′- ccc ccg ctt gag ggc agg, | (SEQ ID NO:18) |
| 5′- cct ctt ttc ccg gtg gag, | (SEQ ID NO:19) |
| 5′- cct ctt ttt ccg gtg gag c, | (SEQ ID NO:20) |
| 5′- cac tcc tct ttt cca atg a, | (SEQ ID NO:21) |
| 5′- cac tcc tct tac ttg gtg, | (SEQ ID NO:22) |
| 5′- tag gtg cca gtc aaa ttt tg, | (SEQ ID NO:23) |
| 5′- ccc ctt ctg atg ggc agg, | (SEQ ID NO:24) |
| 5′- ccc cct ctg atg ggc agg, | (SEQ ID NO:25) |
| 5′- cga ctt cgc aac tcg ttg, | (SEQ ID NO:26) |
| 5′- cga ctt cgc gac tcg ttg, | (SEQ ID NO:27) |
| and | |
| 5′- cga gtt cgc aac tcg ttg. | (SEQ ID NO:28) |

14. The method according to claim 2 for the simultaneous specific detection of coliform bacteria, wherein the oligonucleotide is selected from the group consisting of

| | |
|---|---|
| 5′- gac ccc ctt gcc gaa a, | (SEQ ID NO:29) |
| 5′- atg acc ccc tag ccg aaa, | (SEQ ID NO:30) |
| 5′- ggc aca acc tcc aag tcg ac, | (SEQ ID NO:31) |
| 5′- gga caa cca gcc tac atg ct, | (SEQ ID NO:32) |
| 5′- aca aga ctc cag cct gcc, | (SEQ ID NO:33) |
| 5′- cag gcg gtc tat tta acg cgt t, | (SEQ ID NO:34) |
| 5′- ggc aca acc tcc aaa tcg ac, | (SEQ ID NO:35) |
| 5′- ggc cac aac ctc caa gta ga, | (SEQ ID NO:36) |
| 5′- acc aca ctc cag cct gcc, | (SEQ ID NO:37) |
| 5′- aca aga ctc tag cct gcc, | (SEQ ID NO:38) |
| 5′- ggc ggt cga ttt aac gcg tt, | (SEQ ID NO:39) |
| 5′- ggc ggt cta ctt aac gcg tt, | (SEQ ID NO:40) |
| 5′- ggc ggt cta ttt aat gcg tt, | (SEQ ID NO:41) |
| 5′- agc tcc gga agc cac tcc tca, | (SEQ ID NO:42) |
| 5′- gga aca acc tcc aag tcg, | (SEQ ID NO:43) |
| 5′- gcc aca acc tcc aag tag, | (SEQ ID NO:44) |
| 5′- atg gcc ccc tag ccg aaa, | (SEQ ID NO:45) |
| 5′- g atg acc ccc tag ccg aaa, | (SEQ ID NO:46) |
| and | |
| 5′- aac ctt gcg gcc gta ctc cc. | (SEQ ID NO:47) |

15. The method according to claim 14, wherein the coliform bacteria is of the species Escherichia coli.

16. A method for the detection of bacteria relevant to drinking water in a sample using an oligonucleotide according to claim 1.

17. The method according to claim 16, wherein the oligonucleotide is used for the simultaneous specific detection of bacteria of the genus Legionella, and wherein the oligonucleotide is selected from the group consisting of

| | |
|---|---|
| 5′- cac tac cct ctc cca tac, | (SEQ ID NO:1) |
| 5′- cac tac cct ctc cta tac, | (SEQ ID NO:2) |
| 5′- c cac cac cct ctc cca tac, | (SEQ ID NO:3) |
| 5′- c cac ttc cct ctc cca tac, | (SEQ ID NO:4) |
| 5′- c cac tac cct ctc ccg tac, | (SEQ ID NO:5) |
| 5′- c cac tac cct cta cca tac, | (SEQ ID NO:6) |
| and | |
| 5′- t atc tga ccg tcc cag gtt a. | (SEQ ID NO:7) |

18. The method according to claim 17, wherein the Legionella is of the species L. pneumophila.

19. The method according to claim 16, wherein the oligonucleotide is used for the detection of faecal streptococci, and wherein the oligonucleotide is selected from the group consisting of

| | |
|---|---|
| 5′- ccc tct gat ggg tag gtt, | (SEQ ID NO:8) |
| 5′- ccc tct gat ggg cag gtt, | (SEQ ID NO:9) |
| 5′- tag gtg ttg tta gca ttt cg, | (SEQ ID NO:10) |
| 5′- cac tcc tct ttt tcc ggt, | (SEQ ID NO:11) |
| 5′- c cac ttc tct ttt tcc ggt, | (SEQ ID NO:12) |
| 5′- c cac tct tct ttt tcc ggt, | (SEQ ID NO:13) |
| 5′- c cac tct tct ttt ccc ggt, | (SEQ ID NO:14) |
| 5′- cac aca atc gta aca tcc ta, | (SEQ ID NO:15) |
| 5′- agg gat gaa ctt tcc act c, | (SEQ ID NO:16) |
| 5′- cca ctc att ttc ttc cgg, | (SEQ ID NO:17) |
| 5′- ccc ccg ctt gag ggc agg, | (SEQ ID NO:18) |
| 5′- cct ctt ttc ccg gtg gag, | (SEQ ID NO:19) |
| 5′- cct ctt ttt ccg gtg gag c, | (SEQ ID NO:20) |
| 5′- cac tcc tct ttt cca atg a, | (SEQ ID NO:21) |
| 5′- cac tcc tct tac ttg gtg, | (SEQ ID NO:22) |
| 5′- tag gtg cca gtc aaa ttt tg, | (SEQ ID NO:23) |
| 5′- ccc ctt ctg atg ggc agg, | (SEQ ID NO:24) |
| 5′- ccc cct ctg atg ggc agg, | (SEQ ID NO:25) |
| 5′- cga ctt cgc aac tcg ttg, | (SEQ ID NO:26) |
| 5′- cga ctt cgc gac tcg ttg, | (SEQ ID NO:27) |
| and | |
| 5′- cga gtt cgc aac tcg ttg. | (SEQ ID NO:28) |

20. The method according to claim 16, wherein the oligonucleotide is used for the simultaneous specific detection of coliform bacteria, and wherein the oligonucleotide is selected from the group consisting of

| | |
|---|---|
| 5′- gac ccc ctt gcc gaa a, | (SEQ ID NO:29) |
| 5′- atg acc ccc tag ccg aaa, | (SEQ ID NO:30) |
| 5′- ggc aca acc tcc aag tcg ac, | (SEQ ID NO:31) |
| 5′- gga caa cca gcc tac atg ct, | (SEQ ID NO:32) |
| 5′- aca aga ctc cag cct gcc, | (SEQ ID NO:33) |
| 5′- cag gcg gtc tat tta acg cgt t, | (SEQ ID NO:34) |
| 5′- ggc aca acc tcc aaa tcg ac, | (SEQ ID NO:35) |
| 5′- ggc cac aac ctc caa gta ga, | (SEQ ID NO:36) |
| 5′- acc aca ctc cag cct gcc, | (SEQ ID NO:37) |
| 5′- aca aga ctc tag cct gcc, | (SEQ ID NO:38) |
| 5′- ggc ggt cga ttt aac gcg tt, | (SEQ ID NO:39) |
| 5′- ggc ggt cta ctt aac gcg tt, | (SEQ ID NO:40) |
| 5′- ggc ggt cta ttt aat gcg tt, | (SEQ ID NO:41) |
| 5′- agc tcc gga agc cac tcc tca, | (SEQ ID NO:42) |
| 5′- gga aca acc tcc aag tcg, | (SEQ ID NO:43) |
| 5′- gcc aca acc tcc aag tag, | (SEQ ID NO:44) |
| 5′- atg gcc ccc tag ccg aaa, | (SEQ ID NO:45) |
| 5′- g atg acc ccc tag ccg aaa, | (SEQ ID NO:46) |
| and | |
| 5′- aac ctt gcg gcc gta ctc cc. | (SEQ ID NO:47) |

21. The method according to claim 20, wherein the coliform bacteria is a bacteria of the species Escherichia coli.

22. A kit for performing the method according to claim 2, comprising at least one oligonucleotide according to claim 1.

23. The kit according to claim 22, comprising at least one oligonucleotide in a hybridization solution.

24. The kit according to claim 22, further comprising a washing solution.

25. The kit according to claim 24, further comprising one or more fixation solutions.
